(19) United States
(12) Patent Application Publication
(10) Pub. No.: US 2011/0301120 A1
(43) Pub. Date: Dec. 8, 2011

(54) USE OF A CHITOSAN POLYMER FOR HEPARIN NEUTRALIZATION

(75) Inventors: Maria NOWAKOWSKA, Krakow (PL); Krzysztof SZCZUBIALKA, Krzywaczka (PL); Kamil KAMINSKI, Zawoja (PL); Karolina ZAZAKOWNY, Krakow (PL)

(73) Assignee: UNIWERSYTET JAGIELLONSKI, Krakow (PL)

(21) Appl. No.: 13/206,785

(22) Filed: Aug. 10, 2011

Related U.S. Application Data
(63) Continuation of application No. PCT/PL2010/000012, filed on Feb. 9, 2010.

(30) Foreign Application Priority Data
Feb. 10, 2009 (PL) PL387249

(51) Int. Cl. A61K 31/722; A61P 7/04; A01N 1/02; A61P 7/00

(52) U.S. Cl. 514/55; 435/2

(57) ABSTRACT

Use of a chitosan polymer for direct neutralization of heparin in blood and physiological fluids of mammals. The chitosan polymer may be applied as an intravenous solution or for neutralization of heparin in blood or other physiological fluid taken from a donor.

The present application is a continuation of International Application No. PCT/PL2010/000012, filed on Feb. 9, 2010, which claims priority to Polish Patent Application No. PL387249, filed on Feb. 10, 2009, the contents of each of which are incorporated herein by reference.

The subject of the invention is the application of a chitosan polymer for the neutralization of heparin in blond and other physiological fluids.

Heparin was discovered by McLean almost a century ago and has found clinical applications since 1937. It is the first polysaccharide-based drug which is widely applied in the therapy of humans. Heparin is a complex mixture of highly sulfonated glycosaminoglycans (GAGs) produced and stored in the mast cells of the animals (e.g. in porcine intestines or bovine lungs). It shows the highest density of the negative charge among biological molecules with 2.7 negative charges per disaccharide repeating units. Heparin very strongly inhibits blood coagulation, although only one third of heparin molecules shows anticoagulative properties. Its action is based on increasing of the ability of antithrombin (AT) to deactivate thrombin and Xa factor, the enzymes responsible for blood coagulation. Therefore heparin is a drug of choice in the situations when it is necessary to quickly inhibit coagulation, e.g. during surgical procedures, and particularly to prevent clot formation in the devices used for extracorporeal therapy such as dialysers or oxygenators. It also has many other therapeutical uses, e.g. the treatment of unstable angina pectoris or acute myocardial infarction.

However, administration of heparin is accompanied with many adverse effects, of which the most frequent are bleeding, heparin induced thrombocytopenia (HIT), and osteoporosis.

Therefore, it is often necessary to neutralize or remove heparin from the bloodstream after the therapeutic effect of heparin has been achieved. There are several method of heparin neutralization. Usually it is neutralized by the administration of protamine, a protein introduced to the clinical practice as a heparin antagonist almost simultaneously with heparin (Fischer, A Biochem Zeit 278, 133, 1935). It is characterized by extremely high content of basic amino acids (such as arginine, lysine, and histidine) reaching 80%. Another polymer used to remove heparin is poly-L-lysine (Ma, X., Mohammad, S. F., Kim, S. W. Biotechnology and Bioengineering Volume 40, Issue 4, 5 Aug. 1992, Pages 530-536), which is also used to augument protamine action. Yet another approach to the problem of heparin neutralization its enzymatic degradation using immobilized heparinase (Kolde, H.-J., Pelzer, H., Borzhenskaya, L., Russo, A., Rose, M., Tejidor, L. Hamostaseologie Volume 14, Issue 1, 1994, Pages 37-43).

Unfortunately, the above methods of heparin neutralization may have adverse effects themselves. Protamine, if not neutralized or removed from the bloodstream, may induce adverse effects in about 10% of patients. They may be very serious, and often even lethal and include pulmonary hypertension, arterial hypotension, anaphylactic shock, thrombocytopenia, granulocytopenia, activation of the complement system and cytokine release. Heparin neutralization by the application of protamine is incomplete and accompanied with allergic reactions. On the other hand, poly-L-lysine is still a relatively expensive polymer.

There were many attempts to construct devices for physical removal of heparin, mostly based on the application of immobilized poly-L-lysine (Joseph B. Zwischenberger, MD, Roger A. Vertrees, BA, CCP, Robert L. Brunston, Jr., MD, Weike Tao, MD, Scott K. Alpard, MD, and Paul S. Brown, Jr., MD, The Journal of Thoracic and Cardiovascular Surgery 1998 Volume 115, Number 3; Zwischenberger, J. B., Tao, W., Deyo, D. J., Vertrees, R. A., Alpard, S. K., Shulman, G. Annals of Thoracic Surgery Volume 71, Issue 1, 2001, Pages 270-277). The heparin removal device, described in the above papers is included into patient's bloodstream extracorporeally by venovenous shunt. It performs separation of plasma from which heparin is removed by contact with poly-L-lysine and then plasma is returned back to the patient's bloodstream.

In spite of encouraging results, the experiments on the application of such devices are limited and till now none of them has been introduced into clinical practice.

The method frequently used to avoid complications due to the unbound heparin antagonists is their immobilization on the polymeric supports used in the heparin removal devices.

For example, protamine was supported on the matrix prepared by grafting acrylic polymer on cellulose (Hou, K. C., Roy, S., Zaniewski, R., Shumway, E. Artificial Organs Volume 14, Issue 6, 1990, Pages 436-442) or inside cellulose fibers (Wang, T., Byun, Y., Kim, J.-S., Liang, J., Yang, V. C. International Journal of Bio-Chromatography Volume 6, Issue 2, 2001, Pages 133-149).

It was shown that at the blood flow rate of 100 mL/min the bioreactor constructed removed more than 50% of the administered heparin during 10 minutes.

While fast injection of protamine in dogs results in severe hypotension, the application of a bioreactor containing immobilized protamine did not result in any statistically significant changes in monitored hemodynamical parameters.

Another report describes effective removal of heparin using beads obtained from alginate and poly-L-lysine (M. Sunil Varghese, D. Hildebrandt, and D. Glasser, N. J. Crowther, D. M. Rubin, Artificial Cells, Blood Substitutes, and Biotechnology, 34:419-432, 2006).

It was recently found that chitosan crosslinked with genipin can be used in the devices for extracorporeal removal of heparin (Kamil Kamiński, Karolina Zazakowny, Krzysztof Szczubialka, Maria Nowakowska, Biomacromolecules 2008, 9(11), 3127-3132). This polymer may be applied in the form of microspheres or film as filling in devices for extracorporeal removal of heparin. The polymer in the form of crosslinked microspheres or film cannot be, however, applied intravenously in order to achieve instant anticoagulative effect. Until now protamine is used for this purpose, with all the adverse effects described above.

The purpose of the invention was to develop a method of neutralization of the anticoagulant activity of heparin in blood and physiological fluids.

The subject of the invention is the use of a chitosan polymer having the chemical formula shown in Scheme 1, where R denotes H or —C(O)CH₃ or —CH₂CH(OH)CH₂N(CH₃)₃ group for direct neutralization of heparin in blood and physiological fluids in mammals.

In order to obtain a polymer, where R denotes —CH₂CH(OH)CH₂N(CH₃)₃ group, chitosan is reacted with glycidyltrimethylammonium chloride.

Chitosan polymer may be preferably used as an intravenous solution.

Chitosan polymer is preferably used to neutralize heparin in blood or in other physiological fluid taken from a donor.

The studies on the interaction of chitosan and cationically-modified chitosan with heparin have shown that chitosan interacts with heparin in acidic aqueous solutions. This interaction is stronger for lower values of pH. Thus, in the pH 6 solution the mass of chitosan necessary to completely bind heparin in a complex is about 0.7 of heparin mass. With increasing pH the amount of chitosan necessary to completely remove heparin from the solution increases very quickly. At pH of 7.4 the weight of chitosan required to remove heparin from the solution is about two-fold of that of heparin, while at pH 8.0 about five-fold greater than that of heparin.

In order to increase the effectiveness of formation of aggregates by chitosan with heparin and to increase its solubility at pH 7.4, characteristic of blood, it was cationically-modified with glycidyltrimethylammonium chloride (GTMAC). The modified chitosan obtained is soluble in water at pH 7.4. The efficiency of free heparin removal by cationically-modified chitosan is comparable with the efficiency of protamine and is higher for the polymer with higher degree of substitution. Thus, by applying chitosan with sufficient number of amine groups (i.e. with sufficient deacetylation degree) which may be substituted with GTMAC, the cationically-modified chitosan is obtained with the heparin-binding efficiency equal or higher than of that of protamine, while avoiding the negative consequences characteristic of protamine administration.

Moreover, by using dynamic light scattering measurements it was shown that protamine aggregates with heparin are very polydisperse and their size reach 10 μm, while the diameter of the complexes of heparin with cationically-modified chitosan is much smaller and is about 700 nm. Also, these complexes are more monodisperse comparing to the protamine-heparin complexes. Their smaller size compared to protamine-heparin complexes is a significant advantage in the case of intravenous application.

The subject of the invention was presented in more detail in the embodiments. In the experiments shown in the embodiments the following substances were used: low-molecular-weight chitosan (Ch) (Aldrich), glycidyltrimethylammonium chloride (GTMAC, Fluka, 90%), cationically-modified chitosan of two degrees of substitution with GTMAC, denoted ChGl1 and ChGl2 for polymers with lower and higher degree of substitution, respectively), heparin from bovine intestine, sodium salt (Sigma), protamine (grade X, Sigma), Azure A (Fluka, standard Fluka), potassium chloride (analytical grade, POCh Gliwice), potassium dihydrogen phosphate (analytical grade, POCh Gliwice), disodium hydrogen phosphate (analytical grade, POCh Gliwice), sodium chloride (analytical grade, POCh Gliwice), acetic acid (POCh Gliwice), acetone (analytical grade, CHEMED). Water used in all experiments was distilled twice and purified with Millipore Simplicity System.

The UV spectra were recorded using a diode-array HP 8452A spectrophotometer in quartz cuvettes with 1 cm optical path. The microscopic images were obtained using Nikon TE-2000 fluorescence microscope. The size of aggregates in suspensions was determined using a Zetasizer Nano ZS instrument from Malvern Instruments Ltd.

The results of the measurements are shown in figures, where:

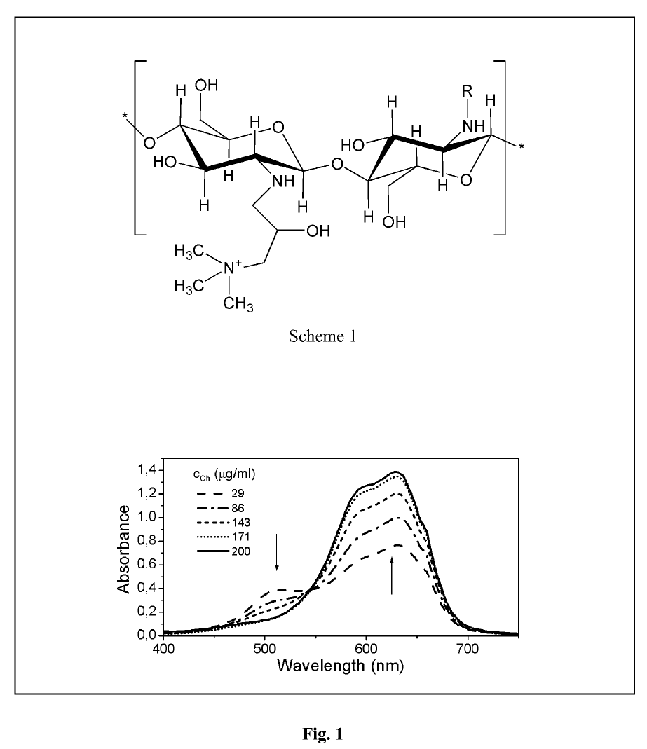

FIG. 1 shows the spectra of Azure A in the presence of heparin (c_{0Hp}=0.196 mg/ml) and chitosan at different concentrations in a pH 5 solution,

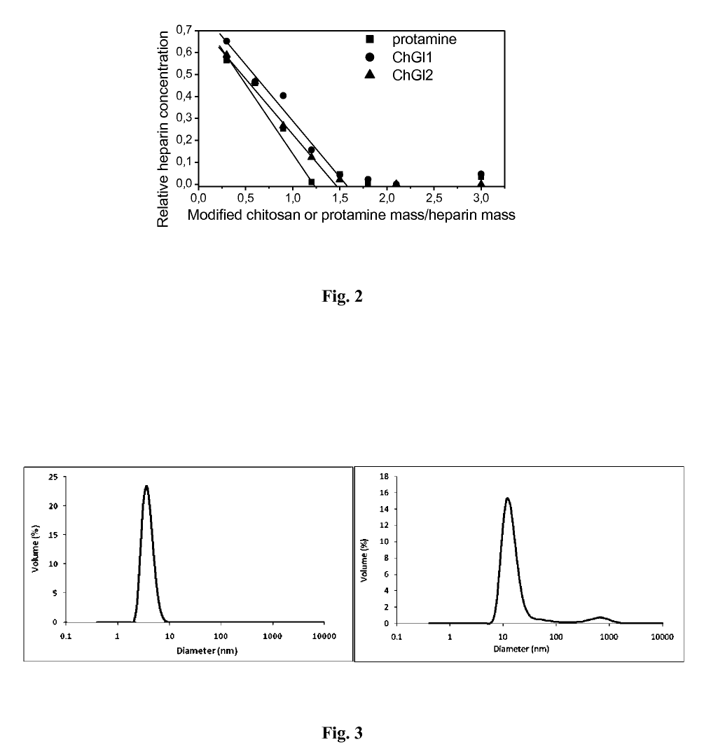

FIG. 2 shows the dependence or a relative heparin concentration (c₀=200 μg/ml) on the ratio of mass of ChGl1, ChGl2 and protamine and heparin where (▪) denotes ChGl1, (♦) denotes heparin, and (▴) denotes ChGl2,

FIG. 3 shows the size of object formed by protamine (left) and ChGl1 (right),

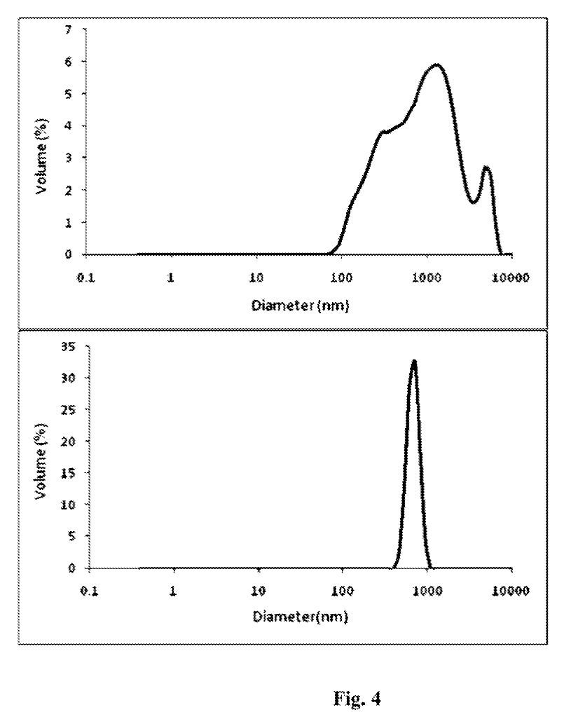

FIG. 4 shows the size of complexes formed by heparin and protamine (left) and ChGl1 (right).

### EXAMPLE 1

### The Studies on the Interaction of Heparin and Chitosan

The concentration of in solution was determined spectrophotometrically using Azure A. 0.9 ml of proper buffer was added to 0.1 ml of heparin solution, followed by addition of 1.0 ml of Azure A solution at 8.0·10⁻⁵ M and the absorption spectrum of the obtained solution was measured. The concentration of heparin was determined on the basis of the intensity of absorption band at 630 nm, which corresponds to monomeric molecules of Azure A. The concentration of free heparin after addition of chitosan was determined by mixing at defined proportions of chitosan solutions in acetic acid. After 10 minutes of energetic shaking the mixtures obtained were centrifuged at 3000 rpm in order to separate the insoluble complex. The concentration of free heparin was determined in the supernatant.

It was shown that addition of heparin to chitosan solution in diluted acetic acid results in its increased turbidity suggesting formation of the heparin-chitosan complex. The absorption spectra of Azure A, a dye used for colorimetric determination of heparin, were recorded in solutions containing increasing concentrations of chitosan after filtering out the formed heparin-chitosan complexes (FIG. 1). The absorption band of Azure A at 630 nm is characteristic of non-associated molecules of the dye, while the band at 513 nm comes from Azure A molecules associated with heparin.

The increase of Azure A absorption at the wavelength of 630 nm with simultaneous decrease of the intensity of absorption at 513 nm due to increased chitosan concentration may prove the decrease of heparin concentration.

The spectrum of Azure A present in the solution of heparin at chitosan concentration of 285 μg/ml is identical with the spectrum of Azure A in the solution of the same concentration of chitosan in the absence of heparin.

This means that at the chitosan concentration of 285 μg/ml or higher free heparin is completely removed from the solution.

### EXAMPLE 2

### Synthesis of Cationically-Modified Chitosan (ChGl)

2.5 g of chitosan was dispersed in 100 ml of distilled water and 10 ml of 0.5% acetic acid was added and mixed for 30 minutes.

In the next step different volumes of glycidyltrimethylammonium chloride (GTMAC) (6.9 ml and 13.8 ml) were added dropwise in order to obtain polymers with different degree of substitution.

The reaction mixture was left for 18 h while mixing with a magnetic stirrer at 55° C.

Then the reaction mixture was centrifuged at 4000 rpm for 10 minutes to remove unreacted polymer.

The supernatant was separated and the product was precipitated with acetone and the suspension was centrifuged at 4000 rpm for 20 min.

The solution was decanted from above the precipitate and the precipitate was pre-dried in air and the dissolved in water.

The solution was centrifuged again as before, and the polymer dissolved in the supernatant was precipitated with a new portion of acetone.

The procedure of dissolving and precipitation was repeated two more times.

The product obtained was dried in a vacuum oven for 24 h.

Two polymers were obtained this way with different degrees of substitution, denoted as ChGl1 and ChGl2, respectively.

### EXAMPLE 3

Interaction of Heparin with Cationically-Modified Chitosan.

The interaction of modified polymers with heparin in the aqueous solution at pH 7.4 was studied (FIG. 2). By measuring heparin concentration with Azure A method it was shown that cationically-modified chitosan effectively complexes free heparin in solution at pH 7.4.

The efficiency of ChGl2, i.e. polymer with a higher degree of substitution with GTMAC, is slightly higher than that of ChGl1.

The efficiency of heparin complexation by both polymers obtained was compared with the efficiency of protamine, a heparin-reversal agent commonly used in medicine (Table 1).

TABLE 1

| | Mass required to bind 1 mg |
|---|---|
| | of heparin in PBS |
| | buffer at pH 7.4 |
| Polymer | [mg] |
| Protamine | 1.2661 |
| ChG11 | 1.5621 |
| ChG12 | 1.4265 |

### EXAMPLE 4

Complexes of Heparin with Protamine and Cationically-Modified Chitosan

Using dynamic light scattering technique the size of objects formed by protamine and ChGl1 in the aqueous solution at pH 7.4 was measured (FIG. 3). The solutions of polymers were prepared in the PBS buffer. The heparin-protamine and heparin-ChGl complexes were obtained by mixing the respective solutions at the volume ratio corresponding to the minimum amount of the polycation needed to completely bind heparin in the solution.

For such mixtures the size and polydispersity of particles were measured.

These measurements have shown that the size of objects formed by protamine is about 4 nm, while the objects formed by ChGl1 are significantly greater—their diameter is about 12 nm.

The diameter of the aggregates of these polymers with heparin was also measured (FIG. 4).

The measurements have shown that the aggregates of protamine with heparin are very polydisperse and their diameter reaches 10 μm, while the diameter of the complexes of heparin with ChGl1 is much smaller—about 700 nm. These complexes are also more monodisperse compared to protamine-heparin complexes.

We claim:

1. The use of a chitosan polymer given with formula given in Scheme 1, 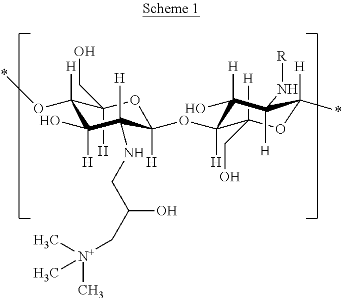
where R denotes H or —C(O)CH₃ or —CH₂CH(OH)CH₂N(CH₃)₃ group, for direct neutralization of heparin in blood and physiological fluids of mammals.

2. Use according to claim 1 wherein the chitosan polymer, where R denotes —CH₂CH(OH)CH₂N(CH₃)₃ is obtained in the reaction of chitosan with glycidyltrimethylammonium chloride.

3. Use according to claim 1 wherein the chitosan polymer is applied in the form of an intravenous solution.

4. Use according to claim 1 wherein the chitosan polymer is applied to neutralize heparin in blood or physiological fluid taken from a donor.

5. A method for direct neutralization of heparin in blood and physiological fluids of mammals comprising contacting the blood or physiological fluid of a mammal with a chitosan polymer of Scheme 1 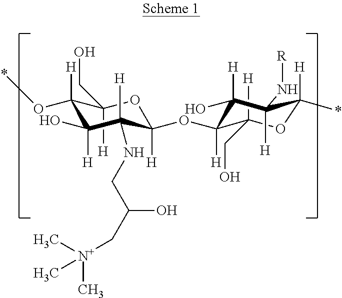
wherein R is H, or —C(O)CH₃ or —CH₂CH(OH)CH₂N(CH₃)₃.

6. The method of claim 5 wherein R is —CH₂CH(OH)CH₂N(CH₃)₃.

7. The method of claim 6, wherein the chitosan polymer is prepared by contacting chitosan with glycidyltrimethylammonium chloride.

8. The method of claim 5, wherein the chitosan polymer is applied in the form of an intravenous solution.

9. The method of claim 5, wherein the chitosan polymer is applied to neutralize heparin in blood or physiological fluid taken from a donor.
